(19) United States
(12) Patent Application Publication
(10) Pub. No.: US 2022/0265542 A1
(43) Pub. Date: Aug. 25, 2022

(54) PETROLATUM ALTERNATIVE FROM RENEWABLE SOURCES

(71) Applicant: Advonex International Corp., Brockville (CA)

(72) Inventor: Chandrashekhar JOSHI, Kingston (CA)

(21) Appl. No.: 17/676,628

(22) Filed: Feb. 21, 2022

Related U.S. Application Data
(60) Provisional application No. 63/151,970, filed on Feb. 22, 2021.

(51) Int. Cl. A61K 8/92; A61K 8/04; A61Q 19/00

(52) U.S. Cl. CPC A61K 8/927; A61K 8/922; A61K 8/042; A61Q 19/007; A61K 2800/5922

(57) ABSTRACT

The present application provides a substitute for petrolatum that does not contain any petroleum-based components. The petrolatum substitute gel composition comprises at least one plant-derived paraffinic wax, at least one plant-derived paraffinic oil, and at least one natural wax. Optionally, the at least one natural wax is beeswax, berry wax (e.g., bay berry wax), candelilla wax, green tea wax, carnauba wax, myrica fruit wax, rice bran wax, a hydrogenated seed oil wax, ouricury wax, shellac wax or tallow tree wax, and the at least one plant-derived paraffinic wax and the at least one plant-derived paraffinic oil are manufactured from a method that comprises a Kolbe-electrolysis process. Also provided is a method for manufacturing the gel composition.

### CROSS-REFERENCE TO RELATED APPLICATION

This application claims the benefit of U.S. Provisional Patent Application Ser. No. 63/151,970, filed on Feb. 22, 2021, which is incorporated by reference herein in its entirety.

### FIELD OF THE INVENTION

The present application pertains to the field of petrolatum alternatives. More particularly, the present application relates to gels comprising plant-derived oils and waxes, and methods of manufacture and uses thereof.

### INTRODUCTION

Petroleum jelly, or petrolatum, which is commonly known as Vaseline® petroleum jelly, is a colloidal system of branched-chain solid hydrocarbons and high-boiling straight-chain liquid hydrocarbons that forms a semisolid viscous mass having a melting point of about 38-56° C. Petrolatum is virtually insoluble in water, alcohol and glycerol. It was developed/discovered in 1859 by the American chemist Robert Chesebrough in Pennsylvania and has been widely used in cosmetics and pharmacy as a salve or cream base. It is still used today in lubricants, cosmetics, sunscreens, lip balms, ink additives, coatings, and the like.

Petrolatum, because of its high viscosity and good solubility, is used as a carrier for other additives and because of its high wax content, is an excellent lubricant. Petrolatum is a by-product of the oil refining process and is produced as a result of de-waxing crude oil. While it is relatively inexpensive, it is on occasion in short supply.

Problems can arise with the use of petrolatum. For example, petrolatum leaves a slight greasy film that can be undesirable in many applications. Also, petrolatum has a relatively narrow range of melt points. Further, there is a shift in the petroleum industry such that a large number of its refining processes are switching over to so-called Group III oils, which are much cleaner and free of pollutants in comparison to previously produced oils. These oils will be used heavily in the manufacture of engine oils and other products that are environmentally sensitive. With this conversion to the Group III oils, the process no longer calls for dewaxing, as the waxes will be broken up and treated by polymers and kept in the oil itself. This means the petrolatum that was once available from refineries producing motor oil base stocks will not be generated or will be generated at a lesser amount.

In cosmetics, there has for many years been a trend toward “green” or “greener” products. An increasing number of consumers wants their cosmetic cleansing and care products to be not only well tolerated and sensorially appealing, but to also be produced from renewable, generally plant-based raw materials that are not only biodegradable but can also be produced in a near-natural and environmentally compatible manner. As a petroleum product, petrolatum does not meet the requirements of environmentally conscious consumers.

Attempts have been made to manufacture alternatives to petrolatum without using petroleum derived waxes or waxes from diminishing sources (see, for example, EP 0 661 924, US 2003/0207971, EP 2 011 483, US 2014/0302161 and US 2019/0298626). However, to date, the formulations developed suffer drawbacks in terms of, for example, formulation characteristics, cost or energy requirements in production and use of diminishing natural sources.

A need remains for a substitute for petrolatum that has the features of petrolatum, but which minimizes its disadvantages.

The above information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present application is to obviate at least some of the disadvantages in the prior art and provide a substitute for petrolatum that does not contain any petroleum-based components. In accordance with an aspect of the present application, there is provided a gel composition comprising at least one plant-derived paraffinic wax, at least one plant-derived paraffinic oil, and at least one natural wax.

In some embodiments, the at least one natural wax is beeswax, berry wax (e.g., bay berry wax), candelilla wax, green tea wax, carnauba wax, myrica fruit wax, rice bran wax, a hydrogenated seed oil wax, ouricury wax, shellac wax or tallow tree wax.

In some embodiments, the at least one plant-derived paraffinic wax and the at least one plant-derived paraffinic oil are manufactured from a method that comprises a Kolbe-electrolysis process.

In accordance with another embodiment, there is provided a method for producing a gel composition as described herein, wherein the method comprises:
a. combining the at least one plant-derived paraffinic wax, the at least one plant-derived paraffinic oil, and the at least one natural wax to form a mixture;
b. heating the mixture until a homogenous liquid is obtained; and
c. cooling the homogenous liquid to form the gel composition.

### BRIEF DESCRIPTION OF TABLES AND FIGURES

For a better understanding of the application as described herein, as well as other aspects and further features thereof, reference is made to the following description which is to be used in conjunction with the accompanying figures, where:

FIG. 1 is a photograph of a formulation containing only a plant-derived wax and a plant-derived oil;

FIG. 2 is a photograph of a formulation containing a plant-derived wax and a plant-derived oil in combination with microcrystalline wax; and

FIG. 3 is a photograph of a formulation according to an embodiment of the present invention containing a plant-derived wax and a plant-derived oil in combination with a natural wax.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As used herein, the term “plant-derived” refers to material that is derived from a plant, or plant part, using a chemical process.

As used herein, the term “natural wax” refers to a wax that is synthesized by an animal or plant. Encompassed within this term is a “modified natural wax,” which is a natural wax that has undergone selective chemical modification to alter one or more properties of the wax. For example, a modified natural wax may be a wax that has been hydrogenated in order to increase its oxidative stability and shelf life.

The present application relates to a gel (or jelly-like) formulation that can be used as an alternative to petrolatum, but that does not include petroleum-based ingredients.

The formulation of the present application comprises at least one plant-derived paraffinic oil, which is combined with at least one plant-derived paraffinic wax. In some embodiments, the plant-derived oils and waxes are characterized by consisting of alkanes of a narrow molecular size distribution (for example, having a single carbon chain length, e.g., C30, or only a narrow range of carbon chain lengths), uniform chemical structure (pure alkanes) and high purity (e.g., having impurities at a level of <10 ppm). For example, such plant-derived oil(s) and wax(es) are prepared using a Kolbe-electrolysis process, such as described in U.S. Pat. Nos. 8,961,775 and 9,611,544, each of which is incorporated herein by reference.

In accordance with certain non-limiting embodiments, the plant-derived oil has a viscosity at 25° C. of from about 15 to about 45 cSt and flash point above 150° C. In certain examples, the plant-derived oil has a viscosity at 25° C. of from about 10 to about 20 cSt and flash point above 150° C. (such as Entrada®-EML from Advonex International) or a viscosity at 25° C. of from about 30 to about 40 cSt and flash point above 200° C. (such as Entrada®-EMH from Advonex International).

In accordance with certain non-limiting embodiments, the plant-derived wax has a melting point of about 40° C. or higher, for example, a melting point in the range of from about 45° C. to about 75° C., such as Entrada®-Wax 51 (melt point: 45-55° C.) or Entrada®-Wax 68 (melt point: 60-75° C.) (both from Advonex International).

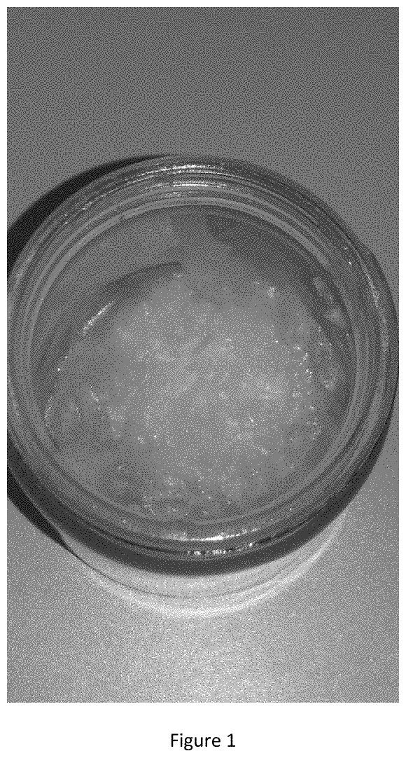

The plant-derived waxes employed in the formulations described herein are relatively hard and brittle. This is a result of result of their narrow molecular size distribution, chemical structure (e.g., pure alkanes) and high purity. Mixing such a plant-derived wax with a plant-derived oil, such as Entrada®-EML or EMH, in various amounts reduced the hardness giving the formulation a gel-like consistency. This mixing was done using the liquid form of the wax, by mixing at temperatures above the melting point of the wax. When cooled, the wax tended to crystallize into small crystals and separate from the oil giving the resulting gel a granular look (see FIG. 1).

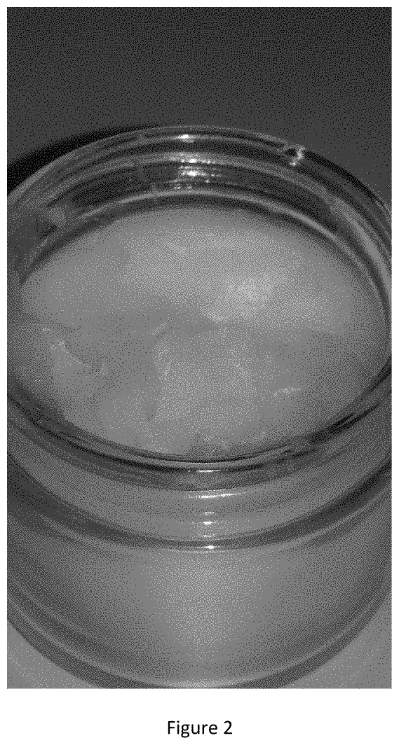

The crystallization problem can be overcome by substituting small amounts of the plant-derived wax with small amounts of microcrystalline wax (MCW). Without wishing to be bound by theory, it is hypothesized that MCW since is a larger weight hydrocarbon molecule is readily mixed with alkanes present in the wax and oil mixture. In practice, it was found that a mixture of 5-20% by weight MCW in the mixture eliminated the granularity observed in the composition with the wax and oil alone (see FIG. 2). However, microcrystalline wax is a petroleum-based product and the goal of the inventor was a formulation that is made entirely from renewable sources.

It was initially thought that since natural waxes consist of esters or hydrogenated triglycerides (both being oxygenated species), they would not mix will with the plant-derived wax and that incorporation of natural waxes in the composition would reduce the shelf-life of the composition. It is known that oxygenated species are hydroscopic and tend to break down chemically due to the presence and adsorption of water over time. However, it has now been, surprisingly found that natural waxes (e.g., floral and seed waxes) indeed are miscible in plant-derived wax.

Accordingly, in the formulations described herein natural waxes, including modified natural waxes, are used instead of MCW and were surprisingly found to also eliminate or minimize crystallization of the plant-derived wax(es) in the resulting formulation. These formulations include at least one natural wax, have a smooth silky texture and consist of 100% non-petroleum (renewable) ingredients. Without wishing to be bound by theory, the natural waxes have a surprising ability to provide some degree of solubilization of the plant-derived wax to thereby interrupt crystallization domains in the plant-derived wax and eliminate or minimize crystallization.

Many different natural waxes (e.g., floral and bio-based) are suitable for use in the formulations of the present application. Natural waxes, including modified natural waxes, suitable for using in the present formulations have a melting point of about 40° C. or more, or of about 45° C. or more. Suitable natural waxes are either plant waxes or animal waxes and include, but are not limited to, beeswax, berry (e.g., bay berry) wax, candelilla wax, green tea wax, carnauba wax, myrica fruit wax, rice bran wax, hydrogenated seed oil waxes (e.g., soywax, palm wax), ouricury wax, shellac wax and tallow tree wax.

In accordance with certain embodiments, the gel formulation of the present application comprises a plant-derived oil or a blend of plant-derived oils at an amount of between about 60% and about 90% by weight of the formulation, or from about 65% to about 85% by weight, or from about 70% to about 80% by weight. The amount of oil included in the formulation can be adjusted depending on the desired softness of the formulation. A softer formulation is made using a higher amount of oil, within the above range, whereas a harder (or firmer) formulation is made using a lower amount of wax, within the above range.

In accordance with certain embodiments, the gel formulation of the present application comprises a plant-derived wax or a blend of plant-derived waxes, and a natural wax or a blend of natural waxes, at a combined amount of between about 10% and about 40% by weight of the formulation, or from about 15% to about 35% by weight, or from about 20% to about 30% by weight. The amount of wax included in the formulation can be adjusted depending on the desired softness of the formulation. A softer formulation is made using a lower amount of wax, within the above range, whereas a harder (or firmer) formulation is made using a higher amount of wax, within the above range. Use of the plant-derived wax at an amount above the recited range results in a formulation that is too waxy and hard (e.g., similar to a lip balm consistency).

In accordance with certain embodiments the gel formulation contains the natural wax or blend of natural waxes in an amount of from about 10% to about 60%, or from about 20% to about 40%, or about 50%, by weight of the combined waxes (i.e., of the combination of the plant derived wax(es) and the natural wax(es)).

The relative amounts of the natural wax to plant-derived wax, and of the wax mixture to the plant-derived oil(s) can be altered to adjust the properties of the formulation, for example to produce a gel with the consistency and smoothness of petrolatum, or to adjust properties as required based on the application of the formulation.

The formulation of the present application can further comprise additives, such as, but not limited to, vitamins, minerals, skin conditioning agents (e.g., humectants such as polyethylene glycol, sorbitols or glycerins), surfactants, opacifiers, colorants, fragrances and the like in amounts effective to accomplish their respective functions.

The present application also provides a method for preparing the formulation in which the plant-derived oil, the plant-derived wax and the natural wax are combined, in the appropriate amounts (e.g., depending on the end use), and heated, with mixing, until a homogeneous liquid mixture is obtained. The mixture is heated to a temperature that enables the solid wax to melt or dissolve into the oils. Once the mixture becomes a homogeneous liquid, the liquid is cooled, with continued mixing, to obtain the gel formulation.

The formulation of the present application is useful on its own as skin care composition or as a component of skin care lotions or creams, in the same manner as petrolatum. Such compositions, lotions or creams are useful in a wide variety of applications, for example, to treat minor scrapes and burns, as a moisturizer, to treat or prevent diaper rash, to prevent skin stains from hair dye or nail polish, to improve or preserve perfume scents, and to treat and prevent pet paw cracks. Alternatively, the present formulation can be used in coatings, lubrication, material finishing, surface cleansing, and similar applications, either on its own or as a component of a composition for these uses.

To gain a better understanding of the invention described herein, the following examples are set forth. It should be understood that these examples are for illustrative purposes only. Therefore, they should not limit the scope of this invention in any way.

### EXAMPLES

### Example 1: Sensory Evaluation Study

Sensory studies were performed to compare petrolatum (Vaseline®) with compositions comprising: (i) a plant-derived wax and a plant-derived oil; (ii) a plant-derived wax, a plant-derived oil and microcrystalline wax; and (iii) and a plant-derived wax, a plant-derived oil and either beeswax or carnauba wax.

A small dab of each sample was placed on the inner forearm of a test subject and applied to skin in 1×1″ area. The texture was evaluated to determine if composition felt smooth while applying the product or if application caused friction or drag on the skin (smoothness test). The texture was also evaluated to determine if the product felt wet or dry during application (oiliness test).

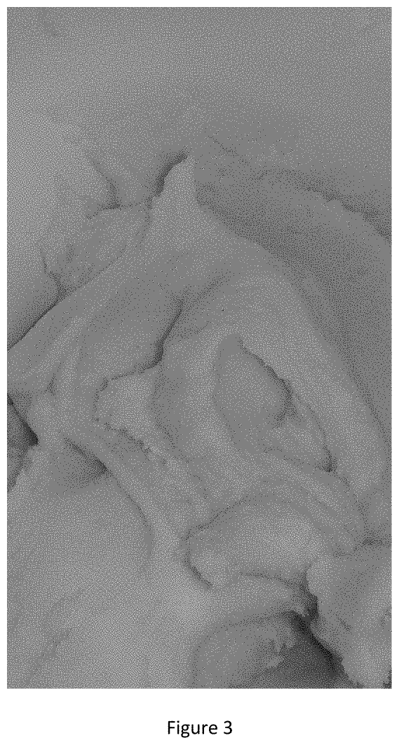

In one formulation, 7 parts Entrada®-EMH was mixed with 1.5 parts Entrada®-WAX 68 and 1.5 parts beeswax and heated to melting above 85° C. The beeswax included a fragrance additive. The resulting solution was mixed well and subsequently cooled, with continued mixing. The result was a smooth silky gel, as depicted in FIG. 3, that felt the same, or very similar to Vaseline®. In contrast, the same composition that did not include the natural wax had a more granular feel. A harder gel was produced by increasing the ratio of wax to liquid.

In a second formulation, 8 parts Entrada®-EMH was mixed with 1 part Entrada®-WAX 68 and 1 part carnauba wax and heated to melting above 90° C. The resulting solution was mixed well and subsequently cooled, with continued mixing. Again, the result a smooth silky gel that felt the same, or very similar to Vaseline®. A harder gel was produced by increasing the ratio of wax to liquid.

The formulation that included Entrada®-EMH, Entrada®-WAX 68 and microcrystalline wax also had a smooth silky feel. However, this was achieved using a petroleum-based product, which is avoided in the formulations of the present application.

Similar formulations can be made substituting Entrada®-WAX 51 for the Entrada®-WAX 68, and Entrada®-EML for the Entrada®-EMH and any of the above-mentioned natural waxes for the beeswax or carnauba wax. In making these substitutions, the proportion of the waxes and liquid may need to be adjusted to create a gel with the desired consistency but this is well known to those skilled in the art of cosmetics formulation.

In all the tested samples, the proportion of the natural (floral or seed) wax could be adjusted to produce a gel having a sensory smoothness similar to petrolatum. These formulations did not feel any oilier than petrolatum.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A gel composition comprising at least one plant-derived paraffinic wax, at least one plant-derived paraffinic oil, and at least one natural wax.

2. The gel composition of claim 1, wherein the at least one natural wax is selected from the group consisting of beeswax, berry wax (e.g., bay berry wax), candelilla wax, green tea wax, carnauba wax, myrica fruit wax, rice bran wax, hydrogenated seed oil waxes, ouricury wax, shellac wax and tallow tree wax.

3. The gel composition of claim 2, wherein the at least one natural wax is beeswax or carnauba wax.

4. The gel composition of claim 1, wherein the at least one plant-derived paraffinic oil is present at an amount of from about 60% to about 90%, or from about 65% to about 85%, or from about 70% to about 80%, by weight of the gel composition.

5. The gel composition of claim 1, wherein the at least one plant-derived paraffinic wax and the natural wax are present at a combined amount of from about 10% to about 40%, or from about 15% to about 35%, or from about 20% to about 30%, by weight of the gel composition.

6. The gel composition of claim 1, wherein the at least one natural wax is present at an amount of from about 10% to about 60%, or from about 20% to about 40%, or about 50%, by weight, based on the combined weight of the at least one plant-derived paraffinic wax and the at least one natural wax.

7. The gel composition of claim 1, wherein the at least one plant-derived paraffinic oil has been manufactured using a Kolbe-electrolysis process.

8. The gel composition of claim 7, wherein the plant-derived paraffinic oil is Entrada®-EML or Entrada®-EMH.

9. The gel composition of claim 1, wherein the at least one plant-derived paraffinic wax has been manufactured using a Kolbe-electrolysis process.

10. The gel composition of claim 9, wherein the plant-derived paraffinic wax is Entrada®-Wax.

11. The gel composition of claim 1, wherein the composition further comprises an additive selected from the group consisting of vitamins, minerals, skin conditioning agents, surfactants, opacifiers, colorants and fragrances.

12. A method for producing a gel composition comprising at least one plant-derived paraffinic wax, at least one plant-derived paraffinic oil, and at least one natural wax, said method comprising:
a. combining the at least one plant-derived paraffinic wax, the at least one plant-derived paraffinic oil, and the at least one natural wax to form a mixture;
b. heating the mixture until a homogenous liquid is obtained; and
c. cooling the homogenous liquid to form the gel composition.

13. The method of claim 12, wherein the at least one natural wax is selected from the group consisting of beeswax, berry wax (e.g., bay berry wax), candelilla wax, green tea wax, carnauba wax, myrica fruit wax, rice bran wax, hydrogenated seed oil waxes, ouricury wax, shellac wax and tallow tree wax.

14. The method of claim 12, wherein the at least one natural wax is beeswax or carnauba wax.

15. The method of claim 12, wherein the at least one plant-derived paraffinic oil is present at an amount of from about 60% to about 90%, or from about 65% to about 85%, or from about 70% to about 80%, by weight of the gel composition.

16. The method of claim 12, wherein the at least one plant-derived paraffinic wax and the natural wax are present at a combined amount of from about 10% to about 40%, or from about 15% to about 35%, or from about 20% to about 30%, by weight of the gel composition.

17. The method of claim 12, wherein the at least one natural wax is present at an amount of from about 10% to about 60%, or from about 20% to about 40%, or about 50%, by weight, based on the combined weight of the at least one plant-derived paraffinic wax and the at least one natural wax.

18. The method of claim 12, wherein the at least one plant-derived paraffinic oil has been manufactured using a Kolbe-electrolysis process.

19. The method of claim 18, wherein the plant-derived paraffinic oil is Entrada®-EML or Entrada®-EMII.

20. The method of claim 12, wherein the at least one plant-derived paraffinic wax has been manufactured using a Kolbe-electrolysis process.

21. The method of claim 20, wherein the plant-derived paraffinic wax is Entrada®-Wax.

22. The method of claim 12, wherein the composition further comprises an additive selected from the group consisting of vitamins, minerals, skin conditioning agents, surfactants, opacifiers, colorants and fragrances.
